(19) United States
(12) Patent Application Publication
(10) Pub. No.: US 2002/0064788 A1
(43) Pub. Date: May 30, 2002

(54) Systematic approach to mechanism-of-response analyses

(76) Inventor: Joseph A. Monforte, Berkeley, CA (US)

Correspondence Address: LAW OFFICES OF JONATHAN ALAN QUINE, P O BOX 458, ALAMEDA, CA, 94501

(21) Appl. No.: 09/909,837

(22) Filed: Jul. 20, 2001

Related U.S. Application Data
(60) Provisional application No. 60/220,080, filed on Jul. 21, 2000.

(51) Int. Cl. C12Q 1/68; G01N 33/53; G01N 33/567; G06F 19/00; G01N 33/48; C12M 1/34

(52) U.S. Cl. 435/6; 702/20; 435/7.1; 435/7.2; 435/287.2

(57) ABSTRACT

The present invention provides methods for identifying new compositions having one or more desired activities, and methods for identifying organisms that are sensitive or resistant to a drug composition. The methods are based upon genetic response profiles generated for an initial set of compositions, where at least one member of the set of compositions has been shown to have at least a first demonstrated activity and a second desired activity. By examining the patterns of genetic and cellular responses (i.e., the genetic response profiles) evoked by a first set of “known” compositions having varying degrees of one or both activities, a preferred pattern of genetic responses can be formulated which corresponds to the desired activity, but not to the demonstrated activity. Additional sets of compounds or compositions can then be screened for the desired genetic response profile, thereby identifying new compositions having the desired activity. Furthermore, populations of organisms can be screened for sensitivity or resistance to drug compositions, based upon comparison of genetic response profiles to the preferred pattern.

### CROSS-REFERENCES TO RELATED APPLICATIONS

This application is related to U.S. provisional patent application No. 60/220,080, filed Jul. 21, 2000 and claims priority to, and benefit of this application, pursuant to 35 U. S. C. §119(e) and any other applicable statute or rule.

### COPYRIGHT NOTIFICATION

Pursuant to 37 C.F.R. 1.71 (e), Applicants note that a portion of this disclosure contains material which is subject to copyright protection. The copyright owner has no objection to the facsimile reproduction by anyone of the patent document or patent disclosure, as it appears in the Patent and Trademark Office patent file or records, but otherwise reserves all copyright rights whatsoever.

### BACKGROUND OF THE INVENTION

Functional genomics is a rapidly growing area of investigation, which includes research into genetic regulation and expression, analysis of mutations that cause changes in gene function, and development of experimental and computational methods for nucleic acid and protein analyses. Proteomics has also emerged as a valuable tool for determining the physiological basis for disease, and for examining the mechanisms of drug action and toxicity. However, with the large numbers of nucleic acid and protein sequences available for examination, selection of biological targets for the development of potential new drug compositions must shift towards technology platforms that can add additional value to the gene selection process, for example, by correlating a particular molecular target with the underlying pathophysiology of a disease. There continues to be a need to identify novel targets and drug compositions that are relevant to disease. The present invention meets these and other needs by providing new methods for identifying compositions having a desired activity, as well as methods for identifying organisms that are sensitive or resistant to drug compositions.

### SUMMARY OF THE INVENTION

The present invention provides methods for identifying new compositions having a desired activity. The methods are based upon genetic response profiles generated for an initial set of compositions, wherein at least one member of the set of compositions has been shown to have at least a first demonstrated activity and a second desired activity. The methods include the steps of providing the first set of compositions, determining a genetic response profile for each member composition, comparing the one or more component responses from the genetic response profile to the first demonstrated activity and second desired activity of each member composition, thereby identifying a pattern of responses correlating to a decrease in the first demonstrated activity and an increase in the second desired activity; and screening a library of test compositions for the pattern of responses.

In these methods, determining the genetic response profiles involves a) providing a plurality of cell lines, b) treating each member of the plurality of cell lines with each member composition of the set of compositions; and c) detecting one or more responses to the member composition. The plurality of cell lines comprises at least one modified cell line which differs from a corresponding parent cell line in either the first demonstrated activity or the second desired activity. Optionally, the plurality of cell lines includes both modified cell lines and parental cell lines. In one embodiment of the present invention, one or more of the cell lines are optimized for the analysis of a particular disease area of interest, such as cancer, inflammation, cardiovascular disease, diabetes, various infectious diseases, proliferative diseases, immune system disorders, or central nervous system disorders.

Optionally, the modified cell line differs from the corresponding parent cell line in the activity or concentration of a selected protein or nucleic acid, for example, in response to the addition of one or more agents or compositions. The plurality of cell lines can also be generated via a genetic selection process, giving rise to one or more cell lines which are, for example, drug resistant.

In a preferred embodiment of the present invention, the set of compounds used to generate the initial genetic response profile includes one or more drug compositions identified for treating the first demonstrated activity. The set of compositions can range, for example, from about 5 to about 50 compositions, or optionally, from about 10 to about 20 compositions. Optionally, the set of compositions includes two or more analogous compounds.

During the generation of the genetic response profile, the cell lines are treated with the member compounds. In one embodiment, treating each member of the plurality of cell lines involves administering varying concentrations of the plurality of compounds, thereby generating a dose-response. The cells are then examined using any of a number of broad scanning techniques, to measure the concentration or activity of at least one gene or gene product, in addition to the desired second activity (and optionally, the demonstrated first activity). For example, for measurement of RNA-type gene products, the broad scanning technique(s) employed can include microarray analysis, differential display, EST screening, or combinations of these techniques. Alternatively, for the measurement of various proteins, the scanning techniques can include 2D-gel electrophoresis, LC mass spectrometry, and various immunoscreening techniques. Proteins of interest include, but are not limited to, signaling proteins, regulatory proteins, pathway specific proteins, and receptor proteins. Optionally, flow cytometry and/or mass spectrometry can be employed, for example, in the detection of various responses.

Detection of responses can also include detecting a change in any number of cellular or physical processes, including, but not limited to, cellular transcriptional activity, cellular translational activity, gene product activity, stability, abundance, compartmentalization, or phenotypic endpoint. For example, assays including, but not limited to, one or more of an RNA transcription assay, a protein expression assay, a binding assay, a protein function assay, a phenotype-based cellular assay, a metabolic assay, a small molecule assay, an ionic flux assay, a reporter gene assay, a cell proliferation assay, an apoptosis assay, a cell adhesion assay, a cell invasion assay, a calcium signaling assay, a cell cycling assay, a nitric oxide signaling assay, a receptor expression assay, or a gene promoter reporter assay, can be employed in the methods of the present invention.

Comparative analysis are performed on the one or more responses, the first demonstrated activity and the second desired activity, to generate a pattern of responses correlating to the first demonstrated activity and the second desired activity. The desired pattern is preferably a decrease in the first demonstrated activity, concomitant with an increase in the desired activity. Alternatively, the first demonstrated activity may stay at the same or similar level, while the desired activity is increased or amplified. Comparative analyses can be approached in any of a number of ways, including, but not limited to, generating a graphical representation of the one or more responses over a plurality of time points, or performing mathematical calculations such as clustering analysis, multivariate analysis, analysis in n-dimensional space, principle component analysis, or difference analysis.

As a further step in the methods of identifying a new composition with a desired activity, a second set of compositions, or library of compositions, is screened by determining the genetic response profiles for member components. Optionally, the genetic profile is determined in a manner similar to that used for the first set of compositions. However, the set of genetic responses determined need not be the same as those determined for the first set of composition; a selected subset of responses can be monitored.

The present invention also provides methods of identifying organisms that are sensitive to treatment with a drug composition. The methods include the steps of: identifying a set of genetic response markers (e.g., a set of genes which correlate to expression response markers) of a biochemical process or disease state for which the drug composition is used as treatment; providing a plurality of cell lines, wherein the plurality of cell lines comprises at least one modified cell line that differs from a corresponding parent cell line in at least one expression marker, or in its sensitivity to the drug composition; determining one or more genetic response profiles by a) treating each member of the plurality of cell lines with the drug composition; and b) monitoring the set of genetic response markers; comparing the one or more genetic response profiles to clinical data for a first population of organisms, thereby identifying a pattern of responses correlating to sensitivity to treatment with the drug composition; and generating additional genetic response profiles for members of a second population of organisms and screening the additional genetic response profiles for the pattern of responses correlating to sensitivity, thereby identifying organisms that are sensitive to treatment with the drug composition. Optionally, the genetic response marker comprises a marker which correlates to drug sensitivity, and the plurality of cell lines includes cell lines which are resistant to the drug treatment. The cell lines can be generated from a subset of cell lines used to identify the set of genes which correlate to the biochemical process (for example, apoptosis) or disease state (e.g., cancer).

As described in greater detail below, the methods provided herein provide mechanisms for the a) determination of the most probable mechanism or mechanisms of action for a drug composition, b) identification of new compositions having a desired activity, and c) identification of organisms that are sensitive (or resistant) to treatment with a drug composition

### DETAILED DISCUSSION

Before describing the present invention in detail, it is to be understood that this invention is not limited to particular compositions or biological systems, which can, of course, vary. It is also to be understood that the terminology used herein is for the purpose of describing particular embodiments only, and is not intended to be limiting. As used in this specification and the appended claims, the singular forms “a”, an and “the” include plural referents unless the content clearly dictates otherwise. Thus, for example, reference to “a device” includes a combination of two or more such devices, reference to “an analyte” includes mixtures of analytes, and the like.

Unless defined otherwise, all technical and scientific terms used herein have the same meaning as commonly understood by one of ordinary skill in the art to which the invention pertains. Although any methods and materials similar or equivalent to those described herein can be used in the practice for testing of the present invention, the preferred materials and methods are described herein.

Definitions

In describing and claiming the present invention, the following terminology will be used in accordance with the definitions set out below.

A “genetic response profile” as used herein refers to a set of responses to a stimuli, reflecting the biochemical events and changes occurring in a cell at a given point in time (i.e. pre- or post-stimulation with, for example, a test composition).

The terms “plurality of cell lines” or “matrix of cell lines” refer to one or more sets of cell lines used, for example, in the preparation of a set of genetic response profiles. Exemplary pluralities of cell lines are described in, for example, PCT application PCT/US01/08670, filed Mar. 16, 2001, which is hereby incorporated by reference in its entirety.

The term “biochemical pathway” is used herein to describe any interrelated series of events or reactions; as such, this term is meant to encompass genetic pathways (series of reactions leading to induction or reduction in gene expression) as well as synthetic or catabolic pathways, metabolic pathways, catalytic pathways and the like.

Methods of Identifying New Compositions with Desired Activities

For many existing and novel therapeutics, the mechanism of cellular response is poorly understood. Even in cases where compounds are known to bind to a specific target, there may be secondary or tertiary binding events that are responsible for the principal in vivo therapeutic mechanism. In addition, one or more secondary effects (e.g. “side effects”) of some therapeutic compounds may constitute an additional desired activity, independent of the demonstrated activity for which the therapeutic compound was initially developed. By understanding how a set of compounds and/or compound analogues effect various genetic and cellular responses in a selected series of cell lines, it is possible to correlate a set of responses with the desired activity (and optionally, without the demonstrated activity), thereby providing a screening mechanism for identifying, selecting, and/or optimizing compositions that produce the desired response profile or target a specific disease area of interest. Furthermore, this approach can be used to evaluate and anticipate the consequences of clinical use of the selected compound(s), information that is potentially valuable for deciding whether or not to carry a compound into the clinic, or in aiding the FDA review process.

The present invention provides methods for identifying new compositions having one or more desired activities. The methods are based upon genetic response profiles generated for an initial set of compositions, where at least one member of the set of compositions has been shown to have at least a first demonstrated activity and a second desired activity. By examining the patterns of genetic and cellular responses (i.e., the genetic response profiles) evoked by a first set of compositions having varying degrees of one or both activities, a preferred pattern of genetic responses can be formulated which corresponds to the desired activity, but not to the demonstrated activity. Additional sets of compounds or compositions can then be screened for the desired genetic response profile. Further aspects of the methods of the present invention are described in greater detail in the following sections.

Cell Lines

The methods of the present invention are based upon responses generated in a plurality of cell lines. The plurality of cell lines includes at least one modified cell line which differs from another cell line, optionally the parent line, in either the first demonstrated activity or the second desired activity. The differences in the cell lines provide the means to identify and dissect one or more responses associated with each activity.

In one embodiment, one or more of the cell lines included in the plurality of cell lines differ in the concentration or activity of only one or a few nucleic acids and/or proteins, optionally leading to an altered activity level for either the first demonstrated activity or the second desired activity. These pin-point differences simplify the process of identifying responses that correlate specifically to one or both activities. In another embodiment, the cell lines differ in the activity of multiple nucleic acids and/or proteins, some of which are associated with the first demonstrated activity and/or the second desired activity, while others are not. The responses generated by these lines can also be used to identify and analyze the specific responses associated with each activity. Additional information can be obtained, for example, from the use of a larger set cell lines, and/or using scientific knowledge available from a number of sources including research databases and publications.

Potential member cell lines includes cell lines derived from, for example, one or more different types of tissues or tumors, primary cell lines, cells which have been subjected to transient and/or stable genetic modification, and the like. Optionally, the cells are mammalian cells, for example murine, rodent, guinea pig, rabbit, canine, feline, primate or human cells. Alternatively, the cells can be of non-mammalian origin, derived, for example, from frogs, amphibians, or various fishes such as the zebra fish.

Cell lines which can be used in the methods of the present invention include, but are not limited to, those available from cell repositories such as the American Type Culture Collection (www.atcc.org), the World Data Center on Microorganisms (http://wdcm.nig.ac.jp), the European Collection of Animal Cell Culture (www.ecacc.org) and the Japanese Cancer Research Resources Bank (http://cellbank.nihs.go.jp). These cell lines include, but are not limited to, HeLa cells, COS cells, lung carcinoma cell lines including squamous cell carcinoma cell lines (such as LK-2, LC-1, EBC-1, and NCI-H157), large cell carcinoma cell lines (such as H460 and H1299), small-cell carcinoma cell lines (such as H345, H82, H209, and N417); adenocarcinoma cell lines (such as A549, H322, H522, H358, H23 and RERF-LC-MS); fibrosarcoma cell lines (such as HT1080); prostrate cancer cell lines (e.g., PC3, DU145, LNCaP, MDA-PCa 2a, MDA-PCa 2b, ARCaP) and other cell lines commonly used by one of skill in the art (for example: 293, 293Tet-Off, CHO-AA8 Tet-Off, MCF7, MCF7 Tet-Off, LNCap, T-5, BSC-1, BHK-21, Phinx-A, 3T3, ZR 75-1, HS 578-T, DBT, Bos, CV1, L-2, RK13, HTTA, HepG2, BHK-Jurkat, Daudi, RAMOS, KG-1, K562, U937, HSB-2, HL-60, MDAHB231, C2C12, HTB-26, HTB-129, HPIC5, A-431, CRL-1573, 3T3L1, Cama-1, J774A.1, HeLa 229, PT-67, Cos7, OST7, HeLa-S, THP-1, and NXA.) Additional cell lines for use in the methods and kits of the present invention can be obtained, for example, from cell line providers such as Clonetics Corporation (Walkersville, Md.; www.clonetics.com).

The number of cell lines employed in the methods of the present invention will vary based upon a number of factors, such as the desired activity, the disease area of interest, and the number of relevant cell lines available. Additional considerations include, but are not limited to, the representation of diverse cell types (for example, the use of diverse cancer cell types for screening of cancer inhibitory compounds), previous usage in the study of similar compounds, and sensitivity or resistance to drug treatment. The plurality of cell lines can range in number from, for example, about two cell lines to about 5, about 10, about 15, about 20, or more cell lines (to as many as about 10³ or about 10⁴ cell lines). Optionally, the methods are performed in a high throughput, multiwell format.

Modified Cell Lines

The plurality of cell lines employed in the methods of the present invention optionally includes both modified cell lines and parental cell lines. The modified cells and optional parental cells typically differ by one or more modifications that have been made to at least one biochemical or genetic pathway. Thus, in some embodiments of the methods of the present invention, the modified cell line differs from the corresponding parent cell line in the activity or concentration of a selected protein or nucleic acid. Alternatively, the differences between parental cell and modified daughter cell may arise from multiple sites or sources of dissimilarity. Any combination of singular-modified cell, multiply modified cell and parental cell can be included in the plurality of cell lines of the present invention.

The difference between modified (daughter) cell line and parental (e.g. wild type) cell line can arise, for example, from changes in the “functional activity” of at least one biological molecule, for example, a protein or a nucleic acid. A difference in the functional activity of a biological molecule refers to an alteration in an activity and/or a concentration of that molecule, and can include, but is not limited to, changes in transcriptional activity, translational activity, catalytic activity, binding or hybridization activity, stability, abundance, transportation, compartmentalization, secretion, or a combination thereof. The functional activity of a biological molecule can also be affected by changes in one or more chemical modifications of that molecule, including but not limited to adenylation, glycosylation, phosphorylation, acetylation, methylation, ubiquitination, and the like.

The alteration in activity or concentration of the at least one biological molecular can arise from a number of treatments of the parental cell line. Furthermore, the alteration can be a permanent change (e.g., a mutation or an irreversible structural modification) or it can be a temporary response to a stimulation. Examples of stimulatory agents, chemicals and treatments which can be used to generate the modified cell lines of the present invention include, but are not limited to, oxidative stress, pH stress, pH altering agents, DNA damaging agents, membrane disrupters, metabolic blocking agents, and energy blockers. Additionally, cellular perturbation may be achieved by treatment with chemical inhibitors, cell surface receptor ligands, antibodies, oligonucleotides, ribozymes and/or vectors employing inducible, gene-specific knock in and knock down technologies.

The identity and use of stimulatory agents, chemicals and treatments are known to one of skill in the art. Examples of DNA damaging agents include, but are not limited to, intercalation agents such as ethidium bromide; alkylating agents such as ethylnitrosourea and methyl methanesulfonate; hydrogen peroxide; UV irradiation, and gamma irradiation. Examples of oxidative stress agents include, but are not limited to, hydrogen peroxide, superoxide radicals, hydroxyl free radicals, perhydroxyl radicals, peroxyl radicals, alkoxyl radicals, and the like. Examples of metabolic blocking and/or energy blocking agents include, but are not limited to, azidothymidine (AZT), ion (e.g. Ca⁺⁺, K⁺, Na⁺) channel blockers, α and β adrenoreceptor blockers, histamine blockers, and the like. Examples of chemical inhibitors include, but are not limited to, receptor antagonists and inhibitory metabolites/catabolites (for example, mavelonate, which is a product of and in turn inhibits HMG-CoA reductase activity).

In some embodiments of the present invention, the alteration in activity or concentration of a biomolecule is evoked in the modified cell in response to the presence of one or more modification agents. Exemplary agents include, but are not limited to, compositions that modify DNA structure (e.g., ethylnitrosourea, quinoxaline antibiotics), compositions that affect DNA activity, compositions that alter protein expression and/or affect protein functional activity (e.g. by inducing or inhibiting the activity), or compositions that induce a combination of these effects. For example, a number of compounds that alter DNA activity do so by inducing or inhibiting transcription or translation of the nucleic acid sequence, or by affecting splicing processes or transcriptional modifications. Alternatively, certain compounds alter protein expression by modifying or interfering with translation, transportation or post-translational modification processes.

Additional agents which can be used to generate modified cell lines include, but are not limited to, antisense agents, ribozymes, receptor ligands (which can either induce or inhibit a range of cellular events), antigens, antibodies, and the like. For example, antisense oligonucleotides can be used to alter gene function, validate gene targets, and even as therapeutic treatments (Baker et al. “Discovery and analysis of antisense oligonucleotide activity in cell culture” Methods 2001 Feb 23:191-8; Koller et al. “Elucidating cell signaling mechanisms using antisense technology” Trends Pharmacol Sci 2000 Apr 21:142-8). Alternatively, ribozymes can be used to down-regulate (by RNA cleavage) or repair (by RNA trans-splicing)gene expression and elicit specific changes in gene/protein expression (see for example, Rossi “Ribozyme therapy for HIV infection” Adv Drug Deliv Rev 2000 Oct 44:71-8; Phylactou “Ribozyme and peptidenucleic acid-based gene therapy” Adv Drug Deliv Rev 2000 Nov 44:97-108). Peptide nucleic acid (PNA) technology can also be used to alter genetic function and produce modified cells for use in the present invention (Nielsen “Peptide nucleic acid: a versatile tool in genetic diagnostics and molecular biology” Curr Opin Biotechnol 2001 Feb;12(1):16-20; Nielsen “Antisense peptide nucleic acids” Curr Opin Mol Ther 2000 Jun;2(3):282-7). Various antibiotics (lexistropsin, luzopeptin, triostin A, distamycin, echinomycin, mitomycin, bleomycin, and other quinoxaline antibiotics), antigens (endotoxins, lectins) and receptor ligands (retinol, estradiol, various growth factors) can also initiate cellular or metabolic changes leading to modified cell lines for use in the present invention.

In one embodiment of the present invention, one or more of the cell lines are optimized for the analysis of a particular disease area of interest prior to use in the plurality of cell lines. Utilization of one or more optimized cell lines or sets of cell lines potentially enhances the screening of compounds for a related treatment. Optionally, the collection of cells can be selected and/or optimized for the analysis of a particular biological or genetic pathway, or for cells that exhibit traits relevant to specific disease phenotypes or areas of interest. Disease areas of interest of the present invention include, but are not limited to, cancer, inflammation, cardiovascular disease, diabetes, infectious disease, proliferative diseases, immune system disorders (such as AIDS), and central nervous system disorders (for example, Alzheimer's disease and Parkinson's disease). However, additional areas of clinical interest could easily be determined by one of skill in the art. If a target molecule for a specific disease is known, the component cell lines in the plurality can be selected for modifications that focus on this particular molecule and the pathways in which it participates. Alternatively, the cell lines can be selected for modifications made in one or more “marker” molecules that correlate to a disease-related pathway of interest.

In some embodiments of the present invention, the plurality of cell lines includes member cell lines which have been generated via a process of genetic selection. Genetic selection, as it is being considered here, is the process of altering the genetic profile, optionally in a directed way, for a cell or whole organism. In one approach, the process typically involves taking the cell through a number of generations of cell cycle. During the replication process genetic mutations occur, either naturally or induced by one or more mutagenic agents (e.g. UV light or a DNA damaging compound, for example, ethyl-nitroso-urea (ENU)). Some of these mutations lead to alteration in the activity or concentration of different RNAs and proteins as monitored in the genetic response profile. Alternatively, mutagenesis can be induced in a more controlled manner (i.e., single nucleotide substitutions, multiple nucleotide substitutions, and insertion or deletion of regions of the nucleic acid sequence), such as by site directed mutagenesis, shuffling, or recursive recombination.

A variety of mutagenesis protocols, such as viral-based mutational techniques, homologous recombination techniques, gene trap strategies, inaccurate replication strategies, and chemical mutagenesis, are available and described in the art. These procedures can be used separately and/or in combination to produce modified cell lines for use in the methods of the present invention. See, for example, Amsterdam et al. “A large-scale insertional mutagenesis screen in zebrafish” Genes Dev 1999 Oct 13:2713-2724; Carter (1986) “Site-directed mutagenesis” Biochem. J. 237:1-7; Crameri and Stemmer (1995) “Combinatorial multiple cassette mutagenesis creates all the permutations of mutant and wildtype cassettes” BioTechniques 18:194-195; Inamdar “Functional genomics the old-fashioned way: chemical mutagenesis in mice” Bioessays 2001 Feb 23:116-120; Ling et al. (1997) “Approaches to DNA mutagenesis: an overview” Anal Biochem. 254(2): 157-178; Napolitano et al. “All three SOS-inducible DNA polymerases (Pol II, Pol IV and Pol V) are involved in induced mutagenesis” EMBO J 2000 Nov 19:6259-6265; and Rathkolb et al. “Large-scale N-ethyl-N-nitrosourea mutagenesis of mice—from phenotypes to genes” Exp Physiol 2000 Nov 85:635-44. Furthermore, kits for mutagenesis and related techniques are also available from a number of commercial sources (see, for example, Stratagene (http://www.stratagene.com/vectors/index2.htm), Clontech (http://www.clontech.com/retroviral/index.shtml), and the Gateway cloning system from Invitrogen (http://www.invitrogen.com). General texts which describe molecular biological techniques useful in the generation of modified cell lines, including mutagenesis, include Berger and Kimmel, Guide to Molecular Cloning Techniques, Methods in Enzymology, volume 152 Academic Press, Inc., San Diego, Calif.; Sambrook et al., Molecular Cloning—A Laboratory Manual (2nd Ed.), volumes 1-3, Cold Spring Harbor Laboratory, Cold Spring Harbor, N.Y., 1989; and Current Protocols in Molecular Biology, F. M. Ausubel et al., eds., Current Protocols, a joint venture between Greene Publishing Associates, Inc. and John Wiley & Sons, Inc., (supplemented through 2000)).

Selection of Modified Cell Lines

The selection process involves the use of different experimental techniques to select those cells which have mutated in the desired manner. For example, the selection process can include, but is not limited to: identifying cells that survive and/or continue to grow under different environments, stresses and/or stimulation; cells that have increased or decreased expression of a particular protein that can be used to sort or separate cells with the altered protein levels, (e.g. using flow cytometry to sort cells that are over expressing a particular cell surface receptor); and cells that have an altered physical phenotype that can be identified and selected, e.g. cells arrested in a particular cycle phase, cells that have altered ability to invade a barrier or translocate, cells that have a different shape, or have or have not differentiated into a different cell type). Numerous additional selection methods are known to one of skill in the art and can be employed to provide cell lines for use in the methods of the present invention.

The plurality of cell lines employed in the methods of the present invention optionally include resistant cell lines. In certain diseases, e.g. cancer, it is as important to understand mechanisms of resistance as well as mechanisms of action of a therapeutic composition. Selection of appropriate cell lines for use in the methods of the present invention will influence both the identification of novel compositions for the treatment (or prevention) of the disease state, as well as any analysis of cellular mechanisms that potentially confer drug resistance. Optionally, one or more existing disease model cell lines (e.g., modified cell lines or parental cell lines) undergo a selection process to create one or more drug resistant cell lines. The resistant cell lines can be analyzed and/or isolated using various techniques known to one of skill in the art; for example, flow cytometry can be used to sort through and collect cells that carry traits of drug resistance. A comparative analysis between non-resistant and resistant cell lines is optionally performed to identify differences in genetic and cellular responses, thereby identifying the cellular elements responsible for resistance. This information can be used, for example, to anticipate potential problems in the clinic, or to design or identify new compounds that bypass these mechanisms of resistance.

As another example, a cell survival selection process can be used to screen for modified cells that have been genetically altered to resist compositions that induce apoptosis. In one approach to generation of apoptosis-resistant cells, a dose response analysis is performed for every member cell line and composition. Concentrations of drugs are tested to identify the optimum dose(s) to maximize killing in a specified length of time, for example, two weeks. Using the optimum dose, cell colonies are treated and selected over a second period of time (e.g., 3 to 4 weeks). Alternatively, modified cell lines can also be generated with varying doses of chemicals. The end product is a series of cell lines with various levels of drug resistance that can be directly compared with their drug sensitive parents.

Knockin, Knockout, and Knockdown Cell Lines

Cell lines carrying specific gene knockdowns or knockins provide excellent model systems for analyzing biochemical and genetic mechanisms, particularly when the only difference among the cell lines is the alteration in the level and/or activity of a single protein or nucleic acid. These pinpoint genetic alterations provide an efficient means to decipher the roles played by various nucleic acids and/or proteins within the biochemical pathways in which they participate.

For example, HeLa cell lines can be finely altered to, in one circumstance, over express the p53 protein, and in another circumstance to under express c-myc. These alterations involve the insertion of exogenous elements that enable the overproduction of a protein (knockin) or reduction in the production of a constitutive protein (knockdown) within the cell. Alternatively, the targeted gene can be prevented from expressing any protein (knockout) via a number of processes, including deletion of the gene or transcription promoting elements for the gene at the DNA level within the cell. Knockout modifications generally involve modification of the gene or genes within the genome (see, for example, Gonzalez (2001) “The use of gene knockout mice to unravel the mechanisms of toxicity and chemical carcinogenesis” Toxicol Lett 120:199-208). Knockdown modifications are typically achieved by either treatment with an exogenous agent (e.g. antisense or ribozyme) or by insertion into the genome of one or more vectors expressing a product that hybridizes to nucleic acid. The target nucleic acid is commonly RNA, although DNA molecules can also be targeted. Furthermore, knockouts can be either heterozygous (e.g. inactivating only one copy of the gene) or homozygous (inactivating both copies of the gene). One exemplary database of mouse knockouts can be found at http://research.bmn.com (the BioMedNet mouse knockout and mutation database).

Knockout modifications generally involve modification of the gene or genes within the genome (see, for example, Gonzalez (2001) “The use of gene knockout mice to unravel the mechanisms of toxicity and chemical carcinogenesis” Toxicol Lett 120:199-208). Knockdown modifications are typically achieved by either treatment with an exogenous agent (e.g. antisense or ribozyme) or by insertion into the genome of one or more vectors expressing a product that hybridizes to nucleic acid. The target nucleic acid is commonly RNA, although DNA molecules can also be targeted. Furthermore, knockouts can be either heterozygous (e.g. inactivating only one copy of the gene) or homozygous (inactivating both copies of the gene). One exemplary database of mouse knockouts can be found at http://research.bmn.com (the BioMedNet mouse knockout and mutation database).

Once a genetic response profile has been developed for a desired activity or biological system, gene-specific knockdowns can be created to specifically perturb principal target molecules within the system. Knockdowns are typically utilized in two ways. The first use is to confirm that a targeted knockdown leads to the same genetic and phenotypic response as is caused by a model or principal compound (e.g., the composition that evokes the first demonstrated activity and the second desired activity). The second common application is the use of stable knockdowns to turn off principal pathways with the cells. These cell are then treated with the compositions and screened to determine which pathways are primary to the phenotypic response stimulated by the compound. A knock down within the key pathway will block the mechanism of action and show an altered genetic response profile, thereby confirming the primary mechanism.

Thus, the plurality of cell lines employed in the present invention can include a combination of parental or wildtype cells, singular-modification cells, multiply-modified cells, resistant cells, cells optimized for a particular disease state, and the like. Further details regarding the generation and use of pluralities of cell lines can be found in PCT application PCT/US01/08670 (Monforte et al.), filed Mar. 16, 2001.

Compositions and Activities

The methods of the present invention include the step of providing a first set of compositions, wherein at least one member of the first set of compositions comprises at least a first demonstrated activity and a second desired activity. In addition, the methods include the step of screening a second set of compositions for the pattern of responses, thereby identifying a new composition with the desired activity. The genetic response profiles generated upon treatment of the plurality of cell lines with the first set of compositions are compared to the first demonstrated activity and second desired activity of each member composition, to identify a desired pattern of responses correlating to an increase in the second desired activity. Preferably, pattern of responses also correlates to a decrease (or at minimum, no change in) the first demonstrated activity.

In a preferred embodiment of the present invention, the set of compounds used to generate the initial genetic response profile includes one or more drug compositions identified for treating the first demonstrated activity. The set of compositions can range, for example, from about 5 to about 50 compositions, or optionally, from about 10 to about 20 compositions.

Optionally, selection of the compounds that are used for generation of the initial genetic response profiles (or for screening of compositions for secondary desired activities) is made based on literature and knowledge of experts in the field of interest. In order to take full advantage of the comparative analysis approach to discerning mechanism of response for a drug or composition and identifying new compositions, it is useful to analyze a selection of compositions including, but not limited to, a range of therapeutics (either approved or currently in clinical trials), therapeutic candidates, research chemicals, libraries of synthetic compositions, natural or biological compounds, herbal compositions, and other chemicals that potentially interact with one or more target molecules or that appear to drive cells to a comparable phenotype(s).

As is appreciated by one skilled in the art, the number of classes of compounds and/or compound analogues (optionally associated with a first demonstrated activity) that can be examined for secondary (desired) activities is extensive, and includes, but is not limited to, the following groups of compounds: ACE inhibitors; anti-inflammatory agents; anti-asthmatic agents; antidiabetic agents; anti-infectives (including but not limited to antibacterials, antibiotics, antifungals, antihelminthics, antimalarials and antiviral agents); analgesics and analgesic combinations; apoptosis inducers or inhibitors; local and systemic anesthetics; cardiac and/or cardiovascular preparations (including angina and hypertension medications, anticoagulants, anti-arrhythmic agents, cardiotonics, cardiac depressants, calcium channel blockers and beta blockers, vasodilators, and vasoconstrictors); chemotherapies, including various antineoplastics; immunoreactive compounds, such as immunizing agents, immunomodulators, immunosuppressives; appetite suppressants, allergy medications, arthritis medications, antioxidants, herbal preparations and active component isolates; neurologically-active agents including Alzheimers and Parkinsons disease medications, migraine medications, adrenergic receptor agonists and antagonists, cholinergic receptor agonists and antagonists, anti-anxiety preparations, anxiolytics, anticonvulsants, antidepressants, antiepileptics, antipsycotics, antispasmodics, psychostimulants, hypnotics, sedatives and tranquilizers, and the like. One advantage to generating genetic response profiles for a defined class of compounds is that the compounds have already been through preclinical and/or clinical evaluation for the demonstrated activity, which provides support for and potentially speeds the process for approval for a second indication (the desired activity).

Genetic Response Profiles

In the methods of the present invention, determining the genetic response profiles involves a) providing a plurality of cell lines, b) treating each member of the plurality of cell lines with a composition; and c) detecting one or more responses to the member composition. The compositions can be a member of the first set of compositions (i.e., during generation of the genetic response profile), or the composition can come from the second set of compositions being screened. Thus, a similar procedure can be employed in screening a library of compositions, although the screening step is not limited to repeating the same process as was previously used to generate the genetic response profiles.

During the generation of the genetic response profile, the cell lines are treated with the member compounds and one or more genetic, biochemical or cellular responses are monitored. For example, changes in any number of cellular or physical processes, including, but not limited to, cellular transcriptional activity, cellular translational activity, gene product activity, stability, abundance, compartmentalization, or phenotypic endpoint, can be included in the genetic response profile. For example, assays including, but not limited to, one or more of an RNA transcription assay, a protein expression assay, a binding assay, a protein function assay, a phenotype-based cellular assay, a metabolic assay, a small molecule assay, an ionic flux assay, a reporter gene assay, a cell proliferation assay, a cell viability assay, an apoptosis assay, a cell adhesion assay, a cell invasion assay, a calcium signaling assay, a cell cycling assay, a nitric oxide signaling assay, a receptor expression assay, or a gene promoter reporter assay, can be employed in the generation of the genetic response profiles of the present invention. The responses can be measured at either a single timepoint or over a plurality of timepoints. Optionally, at least one measurement is collected prior to treatment with the member composition.

The set of genes or gene products selected for inclusion in a given response profile can be selected, for example, by scanning the literature or by performing empirical studies. Preferably, the selected gene or gene products are a) expressed at detectable levels within the plurality of cell lines, and b) are likely to change as a result of exposure to one or more member compositions. Two types of genes (or their respective gene products) are typically monitored during generation of the genetic response profile: genes that are empirical responders (i.e. marker genes) and genes that are known or suspected to be involved in the pathways or disease area of interest. Optionally, one or more genes known to be affected by at least one composition in the set of compositions are monitored (e.g., a positive control). For the sake of experimental efficiency and to optimize the gene set, an initial set of experiments can be performed on both the untreated cell lines and a set of treatments.

RNA and proteins isolated from this small set of samples is analyzed using a number of broad scanning techniques as described below. From this analysis, as well as optional literature data, sets of genes/gene products (e.g. between about 10 and about 20, about 50, about 100 or about 1000) are selected for response profiling. Protein and nucleic acid sequences that can be monitored in the methods of the present invention include, but are not limited to, those listed with the National Center for Biotechnology Information (www.ncbi.nlm.nih.gov) in the GenBank® databases, and sequences provided by other public or commercially-available databases (for example, the NCBI EST sequence database, the EMBL Nucleotide Sequence Database; Incyte's (Palo Alto, Calif.) LifeSeq™ database, and Celera's (Rockville, Md.) “Discovery System”™ database). For example, proteins that can be monitored (e.g., as part of the genetic response profile) in the plurality of cell lines used in the present invention include, but are not limited to, signaling proteins, regulatory proteins, pathway specific proteins, receptor proteins, and other proteins involved in one or more biochemical pathways. Nucleic acids that can be monitored include, but are not limited to, DNA, genomic DNA, BAC or YAC constructs, viral DNA, plasmid DNA or other vectors, tRNA, rRNA, mRNA, guide RNA, snRNA molecules, snoRNA molecules, and hnRNA molecules.

The genetic response profile will be compared to the first demonstrated activity and second desired activity of the member compositions, to generate a desired profile best corresponding to the desired activity. The demonstrated first activity includes any of a number of activities, such as anti-inflammatory, anti-infective, analgesic, anti-hypertensive, antidepressant, immunoreactive, vaso-active and the like. Second desired activities of interest include, but are not limited to, antiproliferative, antineoplastic, or anticancer activity.

Detection Methods

In one embodiment of the present invention, treating each member of the plurality of cell lines involves administering varying concentrations of the plurality of compounds, thereby generating a dose-response. The cells are then examined using any of a number of broad scanning techniques, to measure the concentration or activity of at least one gene or gene product, in addition to the desired second activity (and optionally, the demonstrated first activity).

A number of different detection methods can be used to visualize and monitor the cellular responses as they occur following exposure of the plurality of cell lines to the set of compositions. Such methods include, but are not limited to, RNA transcription assays, protein expression assays, protein function assays, phenotype-based cellular assays, metabolic assays, small molecule assays, ionic flux assays, reporter gene assays, membrane alteration/disruption assays, intercellular signaling assays, selective sensitivity-to-invasion assays, or a combination thereof. Many of these methodologies and analytical techniques can be found in such references as Current Protocols in Molecular Biology, F. M. Ausubel et al., eds., (a joint venture between Greene Publishing Associates, Inc. and John Wiley & Sons, Inc., supplemented through 1999), Enzyme Immunoassay, Maggio, ed. (CRC Press, Boca Raton, 1980); Laboratory Techniques in Biochemistry and Molecular Biology, T.S. Work and E. Work, eds. (Elsevier Science Publishers B.V., Amsterdam, 1985); Principles and Practice of Immunoassays, Price and Newman, eds. (Stockton Press, NY, 1991); and the like.

For example, changes in nucleic acid expression can be determined by polymerase chain reaction (PCR), ligase chain reaction (LCR), Qβ-replicase amplification, nucleic acid sequence based amplification (NASBA), and other transcription-mediated amplification techniques; differential display protocols; microarray analysis, EST screening, analysis of northern blots, enzyme linked assays, and the like. Examples of these techniques can be found in, for example, PCR Protocols A Guide to Methods and Applications (Innis et al. eds) Academic Press Inc. San Diego, Calif. (1990).

Alternatively, the expression pattern of genes can be rapidly analyzed as described by Wang et al. (Nucleic Acids Research (1999) vol. 27, pages 4609-4618). This technique employs PCR amplification of cDNAs which have been cleaved by frequently-cutting endonucleases, such as DpnII and NlaIII, and primed with defined sequences prior to amplification.

Another method for detecting molecular events within the plurality of cell lines utilizes real-time PCR for DNA and rtPCR for RNA, using, for example, FRET (fluorescence resonance energy transfer) in TaqMan® (Applied Biosystems Inc.) or molecular beacon assays. The FRET technique utilizes molecules having a combination of fluorescent labels which, when in proximity to one another, allows for the transfer of energy between labels (see, for example, X. Chen and P. -Y. Kwok, (1997) Nucleic Acid Research vol. 25, pp. 2347-2353).

For the measurement of various proteins, the scanning techniques can include 2D-gel electrophoresis, LC mass spectrometry, and various immunoscreening techniques. Optionally, the responses of the plurality of cell lines can be monitored by fluorescence activated cell sorting, or FACS. A wide variety of flow-cytometry methods have been published. For a general overview of fluorescence activated flow cytometry see, for example, Abbas et al. (1991) Cellular and Molecular Immunology, W.B. Saunders Company; Coligan et al. (eds)(1991) Current Protocols in Immunology, and Supplements, John Wiley and Sons, Inc. (New York); and Kuby (1992) Immunology, W.H. Freeman and Company. Fluorescence activated cell scanning and sorting devices are available from several companies, including, e.g., Becton Dickinson and Coulter.

Alternatively, high throughput screening systems utilizing microfluidic technologies, available, for example, from Agilent/Hewlett Packard (Palo Alto, Calif.) and Caliper Technologies Corp. (Mountain View, Calif.) could be employed for detecting the response(s) generated in the plurality of cell lines. The Caliper Lab Chip™ technology uses microscale microfluidic techniques for performing analytical operations such as the separation, sizing, quantification and identification of nucleic acids (for further information, see www.calipertech.com).

Generation of Profiles

For each cell line and each member composition, a series of experiments can optionally be performed to establish the optimal dosage and time point(s) for measuring response. A dose response study is performed with each compound using one or more of the genetic and/or phenotypic assays described above as the measurable endpoint. Time point(s) and dose level(s) are selected based on these studies.

Observation of cellular events as they occur over time and in response to one or more stimuli provides a dynamic view of the biomolecular activity of the cell. These cellular events, or responses, are evaluated and recorded for comparison. This is achieved by collecting the plurality of data points representing information related to the plurality of cell lines and the one or more responses of the cellular system to the at least one stimulus.

For each experiment performed, the plurality of data points is gathered into a database and used to generate the genetic response profile for the corresponding cell line. The plurality of data points representing the cellular responses upon exposure to the composition being tested can be linear or nonlinear. In one embodiment of the present invention, determining a genetic response profile for each member composition consists of a) selecting a first cell line from the plurality of cell lines; b) evaluating at least one response, and optionally multiple responses; c) recording the evaluation of the at least one response; and d) repeating these steps for additional cell lines in the plurality of cell lines. In another embodiment of the method of the present invention, the evaluating and recording of information is performed on the entire plurality of cell lines simultaneously. During the recording step, the response (or responses) generated for each cell line are entered into a profile database for further analysis. The entire set of cell lines can be evaluated for response to a stimulus, or a subset of the set of cell lines can be examined.

Generation of genetic response profiles for each member composition versus the plurality of cell lines generally results in a large quantity of data reflecting information related to the cell types used and the responses measured for the plurality of cell lines. In one embodiment of the method of the present invention, the plurality of data points is entered as character strings, or as descriptors, into a database. The character strings or descriptors can be used to encode include any relevant information derived from or detected within the plurality of cell lines, including any physical characteristics, activities, or other information related to the cell types used and the responses detected. In general, the database is embodied in a computer or computer readable medium and can be accessed by a user and/or integrated system.

Genetic analysis is optionally complemented with phenotypic analysis of the cells, to build a model of how the cell systems respond to exposure to the set of compositions. A variety of phenotypic data can be acquired during the step of determining a genetic response profile for each member composition of the first set of compositions, including, but not limited to, data related to proliferation, differentiation, apoptosis, cell adhesion, cell invasion, calcium signaling, cell cycling, nitric oxide signaling, receptor expression, gene promoter reporter, cell-cell interaction, cell matrix interaction, cell histology, pathology and other endpoints known to one with skill in the art. The employment of certain types of readout methodologies (e.g. microscopy, flow cytometry, and bioselection) enables partition or selection of subpopulations of cells that can be further profiled for unique traits including altered drug resistance or sensitivity.

Comparative Analyses

Comparative analysis are performed on the one or more responses, the first demonstrated activity and the second desired activity, to generate a pattern of responses correlating to the first demonstrated activity and the second desired activity. The desired pattern is preferably an increase in the desired activity, concomitant with a decrease in the first demonstrated activity. Alternatively, the first demonstrated activity may stay at the same or similar level, while the desired activity is increased or amplified. Comparative analyses can be approached in any of a number of ways, including, but not limited to, generating a graphical representation of the one or more responses over a plurality of time points, or performing mathematical calculations such as clustering analysis, multivariate analysis, analysis in n-dimensional space, principle component analysis, or difference analysis.

Different experimental outcomes are compared by the similarity of the pattern of response profiles generated. This similarity is revealed using, for example, clustering analysis. A number of clustering algorithms are commonly used for this type of study [see Clustering Algorithms, J A Hartigan, Wiley, NY 1975]. The comparisons between profiles can be performed at the level of individual genes, clusters of genes known to be involved in specific pathways or mechanisms, individual cell lines, or for the entire experimental data set. For example, for each experimental pair, e.g. two different composition treatment sets, a distance metric can be defined as D=1−ρ, where ρ is the correlation coefficient between the expression profiles. The value of D indicates the level of similarity between two experimental pairs. In this manner, a matrix can be created wherein chemicals producing similar profiles closely cluster, i.e. D is small, and those with divergent profiles will have large D values. This type of analysis can reveal, for example, similarities in the mechanism of response of various chemicals. Furthermore, analysis among similar cell types and between different cell types is used to determine what cell, tissue, organ or tumor types may be more or less vulnerable when exposed to a given chemical.

In order to ascertain whether the observed changes in response profiles of the treated cell lines are significant, and not just a product of experimental noise or population heterogeneity, an estimate of a probability distribution is optionally constructed for each genetic and phenotypic endpoint in each cell line. Construction of the estimated population distribution involves running multiple independent experiments for each treatment, e.g. all experiments are run in duplicate, triplicate, quadruplicate or the like.

The genetic response information is evaluated and the one or more responses from the genetic response profile are compared to the first demonstrated activity and second desired activity of each member composition. Analysis of the data involves the use of a number of statistical tools to evaluate the measured responses and changes based on type of change, direction of change, shape of the curve in the change, timing of the change and amplitude of change. This information can be used to perceive and interpret the impact that alterations, ranging from a “minor” change in a single nucleotide to major permutations in one or more metabolic pathway, can have on the biological systems network as a whole.

Multivariate statistics, such as principal components analysis (PCA), factor analysis, cluster analysis, n-dimensional analysis, difference analysis, multidimensional scaling, discriminant analysis, and correspondence analysis, can be employed to simultaneously examine multiple variables for one or more patterns of relationships (for a general review, see Chatfield and Collins, “Introduction to Multivariate Analysis,” published 1980 by Chapman and Hall, New York; and Höskuldsson Agnar, “Predictions Methods in Science and Technology,” published 1996 by John Wiley and Sons, New York). Multivariate data analyses are used for a variety of applications involving these multiple factors, including quality control, process optimization, and formulation determinations. The analyses can be used to determine whether there are any trends in the data collected, whether the properties or responses measured are related to one another, and which properties are most relevant in a given context (for example, a disease state). Software for statistical analysis is commonly available, e.g., from Partek Inc. (St. Peters, Mo.; see www.partek.com).

Multivariate statistics is particularly useful for determination and analysis of polygenic effects within a cell line. One common method of multivariate analysis is principal component analysis (PCA, also known as a Karhunen-Loéve expansion or Eigen-XY analysis). PCA can be used to transform a large number of (possibly) correlated variables into a smaller number of uncorrelated variables, termed “principal components.” Multivariate analyses such as PCA are known to one of skill in the art, and can be found, for example, in Roweis and Saul (2000) Science 290:2323-2326 and Tenenbaum et al. (2000) Science 290:2319-2322.

The responses generated by a given plurality of cell lines can be grouped, or clustered, using multivariate statistics. Clusters for each different stimulation (treating) and observation (detecting) experiment are compared and a secondary set of correlations/noncorrelations are made. Based on these different sets of correlations, a network map can be created wherein the relative relationships of the different genetic elements can be established as well as how they may act in concert. In addition, the data can be visualized using graphical representations. Thus, the temporal changes exhibited by the different biochemical and genetic elements within a genetically-related group of cells lines can be transformed into information reflecting the functioning of the cells within a given environment.

Compounds that evoke a similar genetic response are likely to share one or more mechanisms of action. Through analysis of a set of compounds and/or chemical analogues, pathway specific inhibitors and comparable pharmacophores, the mechanistic differences and commonalities can be elucidated. A difference analysis provides the means to identify one or more elements responsible for the desired activity or phenotypic response. In addition, the dose response data coupled with the difference analysis enables the creation of a mechanism of action (MOA) model. Libraries of compositions can be screened for their ability to evoke a genetic response profile similar to that targeted for the desired activity. Furthermore, compositions can be tested against the MOA model to assess if they stimulate similar mechanisms of response.

As a final step in the methods of identifying a new composition with a desired activity, a second set of compositions, or library of compositions, is screened by determining the genetic response profiles for member components. Optionally, the genetic profile is determined in a manner similar to that used for the first set of compositions. However, the number of genetic responses determined need not be the same as those determined for the first set of composition; a selected subset of responses, for example, responses related or correlating to the desired activity being identified, can be monitored.

Additional experimentation can be performed that would aid in the identification of specific genes that, for example, confer sensitivity or resistance to drug treatment. Knowledge of these genes and/or mechanisms can assist in the search for patient segregation markers and surrogate clinical endpoints. As one example, toxicological studies can be performed concomitant with or in addition to screening of compositions for the desired activity.

The following examples are offered for illustration. One of skill in the art will recognize that alternative desired activities can be selected, and a variety of noncritical parameters can be changed.

### EXAMPLE 1

### Development of Chemotherapeutics for Cancer Treatment

The methods of the present invention can be used in the development of novel chemotherapeutics for cancer treatment. The methods employ one or more modified cancer cell lines prepared as follows. One or more cancer cell lines are selected and challenged with a chemotherapeutic agent (e.g. methotrexate or cisplatin), and allowing the cells to grow. Different dosing techniques may be used, for example, increasing the dosage of the agent over multiple cell cycles, using multiple doses of the same concentration over multiple cycles, or just using a single dose of the agent. Modified cells that are capable of growth in the dosed environment are selected. These modified cells have developed a resistance to the particular compound, i.e. they have a different response to the primary activity of the compound versus the parent cell line. Cells that survive the challenge with the chemotherapeutic agent can be individually selected and grown clonally for inclusion in the plurality of cell lines. Optionally, the new cell line is treated with the chemotherapeutic agent to confirm its resistance.

### EXAMPLE 2

### Generation of Apoptosis-modified Cell Lines

The methods of the present invention can also be used to identify novel apoptosis inducers and/or apoptosis inhibitors. For these methods, the plurality of cell lines includes cells that are capable of surviving a pro-apoptosis event. The cells are generated, for example, by treating a cell line with a protein that strongly induces apoptosis, and selecting the cells that survive the treatment. For example, the Fas ligand (which binds to Fas receptor) induces apoptosis in Jurkat cells, a process which can be monitored by flow cytometry. A common apoptosis assay is the Annexin V assay that measures disturbance and inversion of the outer cellular membrane. The vast majority of cells treated with Fas ligand will transition into apoptosis; however, within the cell culture, a small population of cells will resist going into apoptosis. These modified cells can be selectively sorted from the general population using flow cytometry, based on being negative for the Annexin V marker. Alternatively, the modified cells can be selected by subjecting the population to a survival selection screen, such as known to one of skill in the art.

The modified cells have undergone some alteration that prevents the induction of apoptosis. Examples of the types of alterations that may result in survival include mutation of the Fas receptor, strong down regulation of Fas receptor, mutation or down regulation of one of the proteins in the pathway downstream from the receptor, including one of the caspase proteins, or induction of a pathway that is anti-apoptotic with respect to cell regulation. The modified cells are then included in the plurality of cell lines of the methods of the present invention.

### EXAMPLE 3

### Identification of Novel Anti-cancer Compounds Based upon NA+K+-ATPase Inhibitors

Na⁺K⁺-ATPase (sodium pump) is an ion transporter present in the membrane of most eukaryotic cells and either directly or indirectly controls many essential cellular functions (Blanco and Mercer (1998) “Isozymes of the Na-K-ATPase: heterogeneity in structure, diversity in function” Am J Physiol 275:F633-50). For example, Na⁺K⁺-ATPase activity affects intracellular Ca²⁺ levels and modulates gene expression (e.g., androgen receptor) and apoptosis (Bortneret al. (1997) “A primary role for K+ and Na+ efflux in the activation of apoptosis” J Biol Chem 272(51):32436-42; Furuya et al. (1994) “The role of calcium, pH, and cell proliferation in the programmed (apoptotic) death of androgen-independent prostatic cancer cells induced by thapsigargin” Cancer Res 54(23):6167-75), and is modulated by insulin, protein kinases (A, C), cAMP and other second messengers (Haas et al. (2000) “Involvement of Src and epidermal growth factor receptor in the signal-transducing function of Na+/K+-ATPase” J Biol Chem 275(36):27832-7; Huang et al. (1997) “Differential regulation of Na/K-ATPase alpha-subunit isoform gene expressions in cardiac myocytes by ouabain and other hypertrophic stimuli” J Mol Cell Cardiol 29(11):3157-67; Manna et al. (2000) “Oleandrin suppresses activation of nuclear transcription factor-kappaB, activator protein-1, and c-Jun NH2-terminal kinase” Cancer Res 60(14):3838-47; Kometiani et al. (1998) “Multiple signal transduction pathways link Na+/K+-ATPase to growth-related genes in cardiac myocytes: The roles of Ras and mitogen-activated protein kinases” J Biol Chem 273(24):15249-56; Sweeney and Klip (1998) “Regulation of the Na+/K+-ATPase by insulin” Mol Cell Biochem 182:121-33, Xie et al. (1999) “Intracellular reactive oxygen species mediate the linkage of Na+/K+-ATPase to hypertrophy and its marker genes in cardiac myocytes” J Biol Chem 274(27):19323-8). Regulation of this enzyme and its individual isoforms may play a key role in the etiology of some pathological processes including, but not limited to, cardiovascular, neurological, renal, and metabolic diseases purported to involve dysfunction of Na⁺K⁺-ATPase activity (see, for example, Akopyanz et al. (1991) “Tissue-specific expression of Na,K-ATPase beta-subunit” FEBS Lett 289(1): 8-10; Blok et al. (1999) “Regulation of expression of Na+,K+-ATPase in androgen-dependent and androgen-independent prostate cancer” Br J Cancer 81 (1):28-36; McDonough and Farley (1993) “Regulation of Na,K-ATPase activity” Curr Opin Nephrol Hypertens 2(5):725-34; and Rose and Valdes (1994) “Understanding the sodium pump and its relevance to disease” Clin Chem 40(9):1674-85). Furthermore, changes in Na⁺K⁺-ATPase activity may play a role in certain cancers.

The sodium pump is made up of two predominant subunits, a catalytic α subunit and a β subunit that is required for activity. In addition, a third γ subunit has been found in renal cells. The β subunit also functions in cell-cell interactions and in the intracellular transport of the α subunit to the membrane. Each major subunit has several isoforms (e.g., α1, α2, α3, α4 and β1, β2, β3) that show a tissue-specific pattern of expression, which is regulated by the mineralcorticoid and glucocorticoid receptors. For example, the β1-subunit is down-regulated by androgen and increased in androgen insensitive prostate cancer cells.

Inhibition of the Na⁺K⁺-ATPase has an anti-cancer effect in breast cancer clinical studies and various cancer cell lines (Haux (1999) “Digitoxin is a potential anticancer agent for several types of cancer” Med Hypotheses 53(6):543-8). Furthermore, the chromosomal location of the gene encoding the β1 subunit is located in the same region as the prostate cancer sensitivity locus, HPC1. In light of the anticancer activity of Na⁺K⁺-ATPase inhibitors (e.g. a desired effect secondary to the cardiac), Na⁺K⁺-ATPase is a potential cancer drug target. Novel compositions having an increased anticancer activity but with the same or, preferably, a decreased ATPase inhibitory activity, can be identified using the methods of the present invention.

Selection of Initial Set of Compositions

The sodium pump is the only known receptor for the cardiac glycosides, potent inotropic drugs used in the treatment of congestive heart failure (Hauptman and Kelly (1999) “Digitalis” Circulation 99:1265-70). Endogenous ligands structurally similar to digitoxin or ouabain may control the activity of this important molecular complex in vivo. Digitoxin and ouabain have also been implicated as potential anti-cancer drugs based on clinical studies and selective effects on normal versus tumor cells (10, 30, 31, 33). These and related compounds are specific inhibitors of the membrane-bound Na⁺K⁺-ATPase responsible for regulating Na^{+/}K⁺ exchange (and, as a consequence, intracellular Ca²⁺ levels).

Analysis of clinical trial data indicates that five years after mastectomy, women on digitalis had a 9.6-fold reduction in recurrence of breast cancer (Haux, ibid.). It has also been shown that digitalis (30-60 nM) affects cell adherence and induces apoptosis in several Glioblastoma cell lines. The drug tamoxifen also appears to inhibit the Na⁺K⁺-ATPase (in addition to the estrogen receptor, ER) as part of its anti-cancer action (see Repke and Matthes (1994) “Tamoxifen is a Na(+)-antagonistic inhibitor of Na+/K(+)-transporting ATPase from tumour and normal cells” J Enzyme Inhib 8(3):207-12) and is known to have an anti-cancer effect in ER-cancers (e.g., melanoma, glioblastoma).

Androgens are required for prostate development, growth and differentiation, and maintenance of function in the adult. Androgen action is mediated by the androgen receptor (AR), an androgen-dependent transcription factor and member of the nuclear receptor family (which includes receptors to steroids, retinoids, thyroid hormone, and Vitamin D). The AR pathway up-regulates as well as down-regulates numerous factors that affect the growth, differentiation, and survival of prostate epithelial and cancer cells. Androgen insensitivity is one of the major clinical problems in treating prostate cancer (12).

There are several possible functional connections between the Androgen Receptor and the Na⁺K⁺-ATPase. The gene encoding the β-1 subunit of Na⁺K⁺-ATPase is down-regulated in the presence of androgens. Expression is high in androgen-independent cells and low in androgen-dependent cells (grown in the presence of androgens). Down-regulation induced by androgen reduces Na⁺K⁺-ATPase in the membrane. In androgen-dependent cells, a ouabain-induced decrease in Na⁺K⁺-ATPase activity reduces sensitivity of these cells to cisplatin. However, an androgen-induced decrease in Na⁺K⁺-ATPase activity does not protect cells against cisplatin.

Partial inhibition of Na⁺K⁺-ATPase by ouabain increases intracellular Ca²⁺ levels and the expression of c-fos, c-jun, and the transcription factor AP-1. Ca²⁺ mobilizers repress AR-mediated induction of PSA and hKLK2 by inhibiting AR transactivation activity by AP-1 proteins. Androgen deprivation can induce the elevation of intracellular Ca²⁺, the expression of AP-1 genes (c-fos, c-jun), and apoptotic cell death.

Selection of Cell Lines

A number of different cell lines have demonstrated differences in their responsiveness to the describes compositions, their primary activities and apoptosis. For example, digitalis (at non-toxic doses) induces apoptosis in Jurkat (T-cell) and Daudi (B-cell) cell lines, but not in K562 (erthroleukemia cell) lines. Other studies have shown that ouabain sensitizes malignant (but not normal) cells to irradiation (Verheye-Dua and Bohm 1998 “Na+, K+-ATPase Inhibitor, Ouabain Accentuates Irradiation Damage in Human Tumour Cell Lines” Radiation Oncology Investigations 6:109-119).

A number of cell matrices can be selected for their differential response and modeling of prostate cancer. For example, BPH (benign prostatic hyperplasia) cells are commonly used as the “normal” control cell line. PC3 and DU145 cells (parent lines) have lost AR expression and are unresponsive to androgen treatment. In addition they have high doubling times and represent aggressive cancer growth. These same cell lines, if transfected with a vector expressing androgen receptor protein (modified lines), become responsive to androgen treatment.

Complementing the androgen insensitive lines are LNCap, MDA-PCA 2a, 2b, and ARCaP. LNCap expresses AR and is androgen responsive. The MDA-PCa lines overexpress a mutated AR. They have adapted the AR pathway to be able to grow, but with a lower doubling time and are less aggressive than PC3 and DU145. These mutant lines represent loss of activity because of one or more of the following types of adaptations, change in ligand specificity, AR amplification, AR ligand-independent activation, and/or coactivator amplification and co-repressor downregulation. The ARCaP line expresses AR and is growth inhibited upon androgen treatment. This cell line is capable of bypassing the AR pathway for its growth, using one or more of the following mechanisms, activation of other oncogenes or inactivation of tumor suppressor genes (e.g., LNCaP transfected with Ras or Bc1-2), AR mutations and deletions, and/or AR gene inactivation by DNA methylation.

Treatment of these and other like cell lines with the described compositions and possibly others, can be used to generate multiple response profiles and enable the differentiation of activities associated with Na⁺K⁺-ATPase interaction, AR interaction and proapoptotic events. The identified profiles and/or patterns within the response profiles can then be used as target profiles in the screen of compound libraries to identify those compounds with preferred profiles correlating to related proapoptotic activity while minimizing interacting with Na⁺K⁺-ATPase and AR.

### EXAMPLE 4

### Identification of Novel Apoptosis Inducers and Selection of Treatment-sensitive Populations

In addition to identifying novel compositions for treatment of disease states, the genetic response profiles of the present invention can be used to select patients within a population who have a significantly higher probability of responding to treatment with a therapeutic composition. For example, application of cell culture techniques, bioinformatics, and high throughput screening can be used to generate response profiles that predict a probability of clinical efficacy of a drug composition or library of compositions.

The present invention provides methods of identifying organisms that are sensitive to treatment with a drug composition. The methods include the steps of identifying a set of genetic response markers, e.g. one or more genes, RNA sequences, proteins, metabolites, phenotypes and the like, and a correlating genetic response profile for a biochemical process or disease state for which the drug composition is used as treatment; providing a plurality of cell lines, wherein the plurality of cell lines comprises at least one modified cell line which differs from a corresponding parent cell line in its sensitivity to the drug composition; determining a first set of genetic response profiles that potentially indicate drug resistance by a) treating each member of the plurality of cell lines with the drug composition; and b) monitoring the set of genetic response markers; comparing the first set of genetic response profiles to clinical data for a first population of organisms, thereby identifying a pattern of responses correlating to sensitivity to treatment with the drug composition; and generating a second set of genetic response profiles for members of a second population of organisms and screening the second set of genetic response profiles for the pattern of responses correlating to sensitivity, thereby identifying organisms that are sensitive to treatment with the drug composition.

The present example describes the use of genetic response profiles to identify organisms which will respond better to treatment with an apoptosis inducer (AI) (for example, a bisphosphonate class therapeutic composition), using gene expression for multiple genes as the genetic response markers. In brief, a number of genes which correlate to key expression response markers of apoptosis are identified. AI-based genetic response profiles are then determined using an in vitro model of differential response to AI for a plurality of drug-susceptible and drug-resistant cancer cell lines. The genetic response profiles are compared to profiles from clinical samples, to correlate response pattern with clinical outcome. Ultimately, the genetic response patterns are used to analyze patient-derived cells, thereby predicting the likelihood that the patient will respond to treatment with the apoptosis inducer.

Apoptosis and Cancer

Cancer develops through a variety of mechanisms including, but not limited to, the functional failure of multiple gene combinations. Because of the range of genes potentially affected in a given cancer, it is unlikely that any single therapeutic will impact every cancer types. As a consequence, only a portion of a given patient population will preferentially respond to each treatment. It is desirable to model cancer heterogeneity and to visualize how a particular therapeutic affects these cells, linking expression response to phenotypic outcome, and ultimately, clinical outcome. Use of these expression response patterns enables the identification and/or selection of a subset of the patient population with an increased likelihood of response to a particular therapeutic.

One approach to generation of the genetic response profiles is to sample blood and tumor tissue from a large population of cancer patients (>1000) who have been treated with an apoptosis-inducing composition. Generally, it is very difficult and costly to obtain access to the large sample population necessary to capture statistically significant differences attributable to inducer activity, independent of the genetic heterogeneity that naturally occurs among individuals but is unrelated to the disease and treatment. Therefore, an in vitro cell-culture model is employed to generate genetic response profiles and capture many of the statistically significant differences among cancer types. While the cell culture model might not identify all of the possible mechanisms of clinical response, it is likely to be predictive for a large percentage of the population. Likewise, the model can be used to identify those individuals who are unlikely to respond to treatment. Additionally, an in vitro process is far more cost efficient and can be performed quickly while delivering the high level of accuracy in the data necessary for modeling.

Selection of Marker Genes

The first step in the methods of the present invention involves performing experiments to screen for genes that are responsive to AI treatment. These genes include a broad spectrum of gene types, including those that are directly influenced by AI, genes associated with AI response (e.g. apoptosis genes), as well as a number of genes known to play a role in cancer. Optionally, about 1000 genes are screened, to identify the key responders to AI over a variety of cell types. This data will be used to identify the set of genes that correlate to expression response markers of apoptosis.

In one embodiment, the samples are monitored at the RNA level using microarrays. In another embodiment, the samples are analyzed at the protein level using 2-dimensional gel electrophoresis and mass spectrometry.

Approximately 10 different cancer-related cell lines are provided for the study. These lines include cells types that are known in vivo targets for and other AI agents as well as a diversity of potential target tissue types for these therapeutics. Exemplary cancer-related cell lines include: PC-3 (prostate cancer), HepG2 (liver cancer), HL-60 (leukemia), A-549 (lung cancer), MCF-7 (breast cancer), SW620 (colon cancer), Saos-2 (osteosarcoma), MG-63 (osteoblasts), caco-2 (colon cancer), and PA-1 (ovarian cancer).

The cell lines are exposed to the AI, and genes involved in AI response are identified. Cellular and genetic responses are monitored in response to AI treatment for the broad spectrum of cell lines included in the plurality of cell lines. The data (optionally along with other data generated using different chemical compositions for same cell lines) can be used to cluster gene responses and map the genes into a number of categories, including, but not limited to, general expression responders, AI specific responders, disease/cell specific responders, and nonresponders. The identified genes capture the cell response mechanisms for AI treatment. The genes will be used to create an optimal gene set for use in generation of genetic response profiles.

Generation of Genetic Response Profiles and Identification of AI Sensitivity Patterns

The genetic response profiles generated for the cancer lines are used to design the desired expression response pattern that can be used to monitor additional organisms (i.e., patients) and determine a probability of response to AI. Optionally, an in vitro model for mechanisms of AI sensitivity and resistance is prepared using both AI-resistant and AI-sensitive cell lines. The cell lines are generated, for example, from the cell lines analyzed during identification of the genetic response markers. One or more of these cell lines can be used as parent cell lines for the development of multiple resistant daughter lines.

The development of daughter resistant cell lines (modified cell lines) for each parent line involves treatment of parent lines with AI and taking the cells through a selection process. Because the targeted endpoint for susceptibility is cell death, cell survival can be used as a selection tool. Cell lines are treated with AI and surviving cells are cultured. These surviving cells are optionally subjected to 1-2 additional rounds of selection in order to reduce leakage of susceptible cells. From these surviving cells a number of single clones are selected and grown in individual culture. Isolation of single cells and confirmation of their drug resistance is optionally performed by cell sorting flow cytometry. Anywhere from about 10 to about 50 clones are developed and maintained as separate cell lines. One advantage to selecting and using multiple clones is the generation of various modifications leading to resistance (because it is likely that cell survival during treatment will occur through a number of mechanisms). Therefore it is possible to create multiple resistant cell lines that represent several potential resistance mechanisms.

By using genetically-related, parent (sensitive) and daughter (resistant) cell lines representing a number of cancer types, the genes that are specifically responsible for affecting the potency and efficacy of AI can rapidly be determined. Furthermore, the genetic relationship of parent and daughter (modified) cell lines eliminates much of the gene expression variability that is found in unrelated samples, simplifying gene identification, and greatly increasing the correlation between AI and genetic mechanisms that impact its efficacy.

For example, about 2-4 cancer cell lines representative of the cancer types targeted for treatment are selected from the previously tested group, and treated with AI to develop multiple AI resistant lines for each parent cell line. Optionally, a total of about 96 cell lines are generated in this manner. This plurality of cell lines is used to characterize the differential gene expression response in sensitive parent and resistant daughter lines plus and minus exposure to AI. Additionally, a statistical analysis of the expression patterns is performed to identify genes and gene response patterns that indicate the level of AI sensitivity. These experiments provide both a database of expression response patterns for comparative analysis (the first set of genetic response profiles) and the optimal gene set for use in screening patient samples, and for screening and identifying new AI compounds.

For each of the parent and resistant daughter cell lines a gene expression pattern, e.g., a genetic response profile, is determined. The profiles are generated for both AI treated and untreated cultures. Differential parent/daughters expression patterns within each cell line can be determined. A comparison or clustering of different parent/daughter patterns enables a detailed mapping of patterns representative of different mechanisms of resistance. The more parent/daughter patterns generated, analyzed and compared, the higher the level of statistical confidence.

An additional analysis among cell lines can also be performed. These comparisons enable one to visualize consensus patterns that represent resistance mechanisms to AI and to identify resistance mechanisms that may be tissue or cancer-type specific. Conversely, patterns exclusive and universal to parent lines will provide a diagnostic for AI susceptibility. Following this analysis, all of these patterns as represented in a database can be used to evaluate clinical samples in the next step of the methods of the present invention, optionally using the same or similar gene expression tools. In addition, this database may be used to identify new compounds that are AIs but are not susceptible to the same mechanisms of drug resistance.

Clinical Correlation Studies

The methods of the present invention include the step of generating a second set of genetic response profiles for members of a second population of organisms and screening the second set of genetic response profiles for the pattern of responses correlating to sensitivity, thereby identifying organisms that are sensitive to treatment with the drug composition. In one embodiment, the second population of organisms includes clinical samples. A retrospective study to correlate response patterns with clinical outcome assists in the identification of desired patterns of response and in the screening of the second population. The results from screening the second population can also be used to further refine the predictive potential of the pattern analysis.

The methods of the present invention provide a wealth of data, response patterns, methods for obtaining and analyzing samples, and bioinformatic techniques for the analysis of data and determination of therapeutic candidates with improved activity profiles and efficacy probabilities once they are in the preclinical or clinical setting. All of which can be used in an ongoing basis to determine a probability that a drug composition will be effective in treating a disease and each individual patient who has the disease. As a consequence it is fully expected that the genetic response profiles and patterns generated via the methods of the present invention can be used to identify compositions with improved therapeutic characteristics and those individuals with the highest probability of responding to a given drug composition.

Uses of the Methods, Devices and Compositions of the Present Invention

Modifications can be made to the methods and materials as described above without departing from the spirit or scope of the invention as claimed, and the invention can be put to a number of different uses, including:

The use of any method herein, to identify novel compositions.

The use of any method herein, to identify populations which will preferably respond to a composition having a desired activity.

An assay, kit or system utilizing a use of any one of the selection strategies, materials, components, cell matrices, methods or substrates hereinbefore described. Kits will optionally additionally include instructions for performing the methods or assays, packaging materials, one or more containers which contain assay, device or system components, or the like.

In a further aspect, the present invention provides for the use of any component or kit herein, for the practice of any method or assay herein, and/or for the use of any apparatus or kit to practice any assay or method herein.

While the foregoing invention has been described in some detail for purposes of clarity and understanding, it will be clear to one skilled in the art from a reading of this disclosure that various changes in form and detail can be made without departing from the true scope of the present invention. For example, all the methods and compositions described above may be used in various combinations. All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods, and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims. All publications, patents, patent applications, Internet citations, and/or other documents cited in this application are incorporated by reference in their entirety for all purposes to the same extent as if each individual publication, patent, patent application, Internet citation and/or other document were individually indicated to be incorporated by reference for all purposes.

What is claimed is:

1. A method of identifying a new composition with a desired activity, the method comprising:
providing a first set of compositions, wherein at least one member of the first set of compositions comprises at least a first demonstrated activity and a second desired activity;
determining a genetic response profile for each member composition of the first set of compositions by a) providing a plurality of cell lines, wherein the plurality of cell lines comprises at least one modified cell line which differs from a corresponding parent cell line in either the first demonstrated activity or the second desired activity; b) treating each member of the plurality of cell lines with each member composition of the first set of compositions; and c) detecting one or more responses to the member composition;
comparing the one or more responses from the genetic response profile to the first demonstrated activity and second desired activity of each member composition, thereby identifying a pattern of responses correlating to a decrease in the first demonstrated activity and an increase in the second desired activity; and
screening a second set of compositions for the pattern of responses, thereby identifying a new composition with the desired activity.

2. The method of claim 1, wherein the modified cell line differs from the corresponding parent cell line in the activity or concentration of a selected protein or nucleic acid.

3. The method of claim 2, wherein the activity or concentration of a selected protein is altered in response to an addition of one or more agents to the parent cell line.

4. The method of claim 3, wherein the one or more agents comprise compositions that modify DNA structure, alter DNA activity, alter protein expression, inhibit protein functional activity, induce protein functional activity, or combinations thereof.

5. The method of claim 4, wherein the compositions that alter DNA activity or alter protein expression comprise transcription inducers, transcription inhibitors, translation inducers, translation inhibitors, compositions that alter post-transcription modification, compositions that alter splicing, or compositions that alter transportation.

6. The method of claim 4, wherein the one or more agents comprise one or more antisense agents, ribozymes, protein ligands, growth factors, antibodies, antigens, antibiotics, transcription inhibitors, transcription enhancers, translation inhibitors, or translation enhancers.

7. The method of claim 1, wherein providing the plurality of cell lines comprises performing a genetic selection.

8. The method of claim 1, wherein the at least one modified cell line comprises a cell line that is drug resistant.

9. The method of claim 1, wherein providing the set of compounds comprises providing one or more drug compositions identified as a treatment for the first demonstrated activity.

10. The method of claim 1, wherein the second desired activity comprises an antiproliferative activity.

11. The method of claim 1, wherein the second desired activity comprises an antineoplastic activity.

12. The method of claim 1, wherein the first or second set of compositions comprises between about 5 and about 50 compositions.

13. The method of claim 1, wherein the first or second set of compositions comprises between about 10 and about 20 compositions.

14. The method of claim 1, wherein the first or second set of compositions comprises one or more compound analogs.

15. The method of claim 1, wherein providing the plurality of cell lines comprises providing cell lines derived from different types of tissues or tumors, primary cell lines, genetically-modified cell lines, or combinations thereof.

16. The method of claim 1, wherein providing the plurality of cell lines comprises providing target-specific modified cell lines and parent cell lines.

17. The method of claim 1, wherein the plurality of cell lines comprises about two to about ten cell lines.

18. The method of claim 1, wherein the plurality of cell lines comprises cell lines optimized for the analysis of a particular disease area of interest.

19. The method of claim 18, wherein the particular disease area of interest comprises cancer, inflammation, cardiovascular disease, diabetes, an infectious disease, a proliferative disease, an immune system disorder, or a central nervous system disorder.

20. The method of claim 1, wherein one or more cell lines of the plurality of cell lines are selected from the group consisting of: PC3, DU145, LNCaP, MDA-PCa 2a, MDA-PCa 2b, ARCaP, 293, 293Tet-Off, CHO-AA8 Tet-Off, MCF7, MCF7 Tet-Off, LNCap, T-5, BSC-1, BHK-21, Phinx-A, 3T3, HeLa, PC3, DU145, ZR 75-1, HS 578-T, DBT, Bos, CV1, L-2, RK13, HTTA, HepG2, BHK-Jurkat, Daudi, RAMOS, KG-1, K562, U937, HSB-2, HL-60, MDAHB231, C2C12, HTB-26, HTB-129, HPIC5, A-431, CRL-1573, 3T3L1, Cama-1, J774A.1, HeLa 229, PT-67, Cos7, OST7, HeLa-S, THP-1, and NXA.

21. The method of claim 1, wherein treating each member of the plurality of cell lines comprises administering varying concentrations of the plurality of compounds, thereby generating a dose-response.

22. The method of claim 1, wherein detecting the one or more responses comprises performing one or more broad scanning techniques and measuring the concentration or activity of at least one gene or gene product in the plurality of cell lines.

23. The method of claim 22, wherein the gene product comprises RNA and the one or more broad scanning techniques comprise microarray analysis, differential display, EST screening, or combinations thereof.

24. The method of claim 22, wherein the gene product comprises protein and the one or more broad scanning techniques comprise 2D-gel electrophoresis, LC mass spectrometry, immunoscreening techniques, or combinations thereof.

25. The method of claim 1, wherein detecting the one or more responses comprises detecting a change in cellular transcriptional activity, cellular translational activity, gene product activity, stability, abundance, compartmentalization, phenotypic endpoint or a combination thereof.

26. The method of claim 1, wherein detecting the one or more responses comprises performing an RNA transcription assay, a protein expression assay, a binding assay, a protein function assay, a phenotype-based cellular assay, a metabolic assay, a small molecule assay, an ionic flux assay, a reporter gene assay, a cell proliferation assay, an apoptosis assay, a cell adhesion assay, a cell invasion assay, a calcium signaling assay, a cell cycling assay, a nitric oxide signaling assay, a receptor expression assay, a gene promoter reporter assay, or a combination thereof.

27. The method of claim 22, wherein the gene product comprises one or more proteins selected from the group: signaling proteins, regulatory proteins, pathway specific proteins, and receptor proteins.

28. The method of claim 1, wherein detecting the one or more responses comprises performing flow cytometry.

29. The method of claim 1, wherein detecting the one or more responses comprises performing mass spectrometry.

30. The method of claim 1, wherein comparing the one or more responses comprises performing a comparative analysis on the one or more responses, the first demonstrated activity and the second desired activity.

31. The method of claim 30, wherein performing a comparative analysis comprises generating a graphical representation of the one or more responses over a plurality of time points.

32. The method of claim 30, wherein performing a comparative analysis comprises performing one or more techniques selected from the group consisting of: clustering analysis, multivariate analysis, analysis in n-dimensional space, principle component analysis, and difference analysis.

33. The method of claim 1, wherein screening the second set of compositions comprises screening a library of compositions.

34. The method of claim 1, wherein screening the second set of compositions comprises determining a genetic response profile for one or more members of the library of test compositions by:
treating each member of the plurality of cell lines with a member composition of the library of test compositions; and
detecting one or more responses to the member composition.

35. The method of claim 34, wherein the one or more responses collected for the genetic response profiles of the second set of compositions comprises a subset of the responses collected for the genetic response profiles of the first set of compositions.

36. A method of identifying one or more organisms that are sensitive to treatment with a drug composition, the method comprising:
identifying a set of genetic response markers of a biochemical process or disease state for which the drug composition is used as treatment;
providing a plurality of cell lines, wherein the plurality of cell lines comprises at least one modified cell line that differs from a corresponding parent cell line in a sensitivity to the drug composition;
determining one or more genetic response profiles by a) treating each member of the plurality of cell lines with the drug composition; and b) monitoring the set of genetic response markers;
comparing the one or more genetic response profiles to clinical data for a first population of organisms, thereby identifying a pattern of responses correlating to sensitivity to treatment with the drug composition; and
generating additional genetic response profiles for members of a second population of organisms and screening the additional genetic response profiles for the pattern of responses correlating to sensitivity, thereby identifying one or more organisms that are sensitive to treatment with the drug composition.
